(19) United States
(12) Patent Application Publication
(10) Pub. No.: US 2007/0161835 A1
(43) Pub. Date: Jul. 12, 2007

(54) Liquid phase alkylation system

(75) Inventors: James R. Butler, League City, TX (US); Kevin P. Kelly, Friendswood, TX (US)

Correspondence Address: David J. Alexander, P.O. Box 674412, Houston, TX, 77167, US

(73) Assignee: Fina Technology, Inc., Houston, TX (US)

(21) Appl. No.: 11/326,633

(22) Filed: Jan. 7, 2006

(51) Int. Cl. C07C 2/64; C07C 15/067; B01J 8/04; B01J 8/02

(52) U.S. Cl. 585/446; 422/188

(57) ABSTRACT

Alkylation systems and alkylation and regeneration methods are generally described herein. For example, embodiments of the invention generally include an alkylation system, such alkylation system including a plurality of reaction vessels, each reaction vessel adapted to receive at least a portion of an alkylation input stream and contacting the portion of the alkylation input stream with an alkylation catalyst to form a second aromatic compound, wherein the reaction vessels are adapted for liquid phase alkylation. The input stream generally includes a first aromatic compound and the second input stream generally includes a second aromatic compound.

### FIELD

Embodiments of the present invention generally relate to alkylation of aromatic compounds.

### BACKGROUND

Alkylation reactions generally involve contacting a first aromatic compound with an alkylation catalyst to form a second aromatic compound. While various phase conditions may be employed in the alkylation process, liquid phase conditions may be capable of minimizing the yield of undesirable by-products from the alkylation reactor. Unfortunately, liquid phase reaction systems generally have limited options for catalyst regeneration and maintenance. For example, when alkylation systems are utilized in conjunction with dehydrogenation processes, the alkylation system maintenance may be limited to the maintenance time period of the dehydrogenation system (e.g., maintenance every three years.)

Unfortunately, alkylation catalyst systems generally experience deactivation requiring either regeneration or replacement. Additionally, alkylation processes generally require periodic maintenance. Both circumstances generally produce disruptions for liquid phase alkylation processes.

Therefore, a need exists to develop an alkylation system that is capable of reducing catalyst depletion while providing a system adapted for routine maintenance with minimal production disruption.

### SUMMARY

Embodiments of the present invention generally include alkylation systems and alkylation and regeneration methods. For example, embodiments of the invention generally include an alkylation system, such alkylation system including a plurality of reaction vessels, each reaction vessel adapted to receive at least a portion of an alkylation input stream and contacting the portion of the alkylation input stream with an alkylation catalyst to form a second aromatic compound, wherein the reaction vessels are adapted for liquid phase alkylation. The input stream generally includes a first aromatic compound and the second input stream generally includes a second aromatic compound.

In another embodiment, the alkylation system includes an alkylation system adapted to simultaneously alkylate a plurality of input streams under liquid phase conditions, wherein the input streams include an aromatic compound.

Another embodiment generally includes an alkylation method. The alkylation method generally includes contacting a plurality of input streams with an alkylation catalyst disposed within an alkylation system to form an output stream, wherein the input streams includes a first aromatic compound and an alkylating agent and the output stream includes a second aromatic compound and wherein the first aromatic compound remains in the liquid phase throughout the alkylation system.

Embodiments of the invention further include a regeneration method. The regeneration method generally includes regenerating a first alkylation catalyst, wherein the first alkylation catalyst was disposed within an alkylation system prior to regeneration and simultaneously reacting an input stream with a second alkylation catalyst to form an output stream within the alkylation system, wherein the reaction is in the liquid phase.

### BRIEF DESCRIPTION OF DRAWINGS

FIG. 1 illustrates an alkylation/transalkylation process.

FIG. 2 illustrates an embodiment of an alkylation process.

FIG. 3 illustrates an embodiment of an alkylation reaction vessel.

### DETAILED DESCRIPTION

### Introduction and Definitions

A detailed description will now be provided. Each of the appended claims defines a separate invention, which for infringement purposes is recognized as including equivalents to the various elements or limitations specified in the claims. Depending on the context, all references below to the “invention” may in some cases refer to certain specific embodiments only. In other cases it will be recognized that references to the “invention” will refer to subject matter recited in one or more, but not necessarily all, of the claims. Each of the inventions will now be described in greater detail below, including specific embodiments, versions and examples, but the inventions are not limited to these embodiments, versions or examples, which are included to enable a person having ordinary skill in the art to make and use the inventions, when the information in this patent is combined with available information and technology.

Various terms as used herein are shown below. To the extent a term used in a claim is not defined below, it should be given the broadest definition persons in the pertinent art have given that term as reflected in printed publications and issued patents. Further, unless otherwise specified, all compounds described herein may be substituted or unsubstituted and the listing of compounds includes derivatives thereof.

The term “activity” refers to the weight of product produced per weight of the catalyst used in a process per hour of reaction at a standard set of conditions (e.g., grams product/gram catalyst/hr).

The term “conversion” refers to the percentage of input converted.

The term “deactivated catalyst” refers to a catalyst that has lost enough catalyst activity to no longer be efficient in a specified process.

The term “recycle” refers to returning an output of a system as input to either that same system or another system within a process. The output may be recycled to the system in any manner known to one skilled in the art, for example, by combining the output with the input stream or by directly feeding the output into the system. In addition, multiple input streams may be fed to a system in any manner known to one skilled in the art.

The term “regenerated catalyst” refers to a catalyst that has regained enough activity to be efficient in a specified process. Such efficiency is determined by individual process parameters.

The term “regeneration” refers to a process for renewing catalyst activity and/or making a catalyst reusable after its activity has reached an unacceptable level. Examples of such regeneration may include passing steam over a catalyst bed or burning off carbon residue, for example.

Embodiments of the invention generally relate an alkylation system including a plurality of reaction vessels adapted for liquid phase alkylation of a first aromatic compound to form a second aromatic compound.

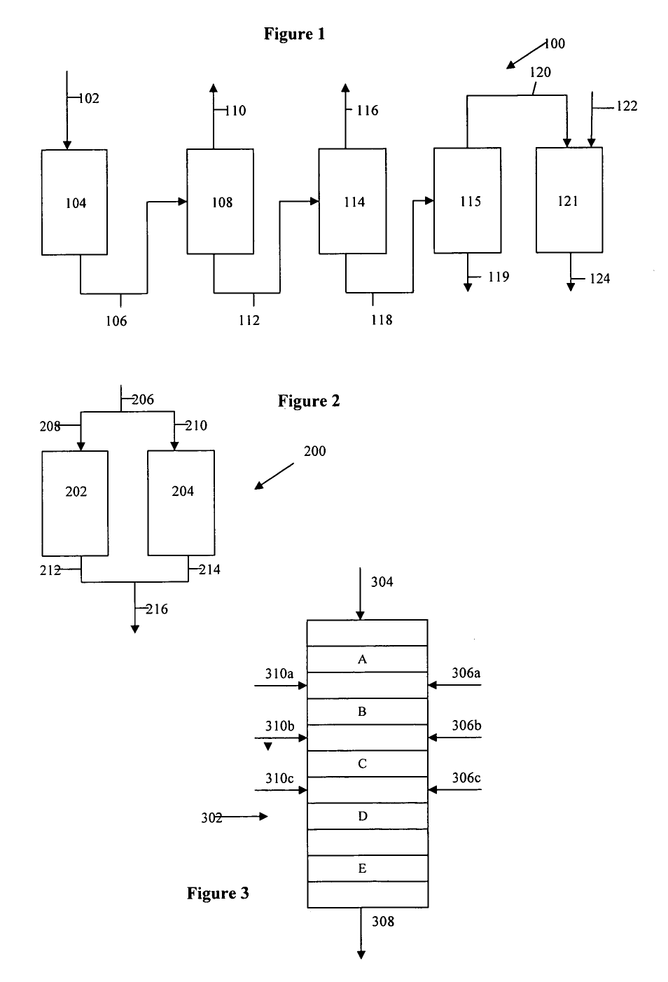

FIG. 1 illustrates a schematic block diagram of an embodiment of an alkylation/transalkylation process 100. The process 100 generally includes supplying an input stream 102 to an alkylation system 104. The alkylation system 104 is generally adapted to contact the input stream 102 with an alkylation catalyst to form an alkylation output stream 106.

At least a portion of the alkylation output stream 106 passes to a first separation system 108. An overhead fraction is generally recovered from the first separation system 108 via line 110 while at least a portion of the bottoms fraction is passed via line 112 to a second separation system 114.

An overhead fraction is generally recovered from the second separation system 114 via line 116 while at least a portion of a bottoms fraction is passed via line 118 to a third separation system 115. A bottoms fraction is generally recovered from the third separation system 115 via line 119 while at least a portion of an overhead fraction is passed via line 120 to a transalkylation system 121. In addition to the overhead fraction 120, an additional input, such as additional aromatic compound, may be supplied to the transalkylation system 121 via line 122 to contact the transalkyation catalyst, forming a transalkylation output 124.

Although not shown herein, the process stream flow may be modified based on unit optimization so long as the modification complies with the spirit of the invention, as defined by the claims. For example, at least a portion of any overhead fraction may be recycled as input to any other system within the process. Also, additional process equipment, such as heat exchangers, may be employed throughout the processes described herein and such placement is generally known to one skilled in the art.

Further, while described below in terms of primary components, the streams indicated below may include any additional components as known to one skilled in the art.

The input stream 102 generally includes an aromatic compound and an alkylating agent. The aromatic compound may include substituted or unsubstituted aromatic compounds. If present, the substituents on the aromatic compounds may be independently selected from alkyl, aryl, alkaryl, alkoxy, aryloxy, cycloalkyl, halide and/or other groups that do not interfere with the alkylation reaction, for example. Examples of substituted aromatic compounds generally include toluene, xylene, isopropylbenzene, normal propylbenzene, alpha-methylnaphthalene, ethylbenzene, mesitylene, durene, cymene, butylbenzene, pseudocumene, o-diethylbenzene, m-diethylbenzene, p-diethylbenzene, isoamylbenzene, isohexylbenzene, pentaethylbenzene, pentamethylbenzene, 1,2,3,4-tetraethylbenzene, 1,2,3,5-tetramethylbenzene, 1,2,4-triethylbenzene, 1,2,3-trimethylbenzene, m-butyltoluene, p-butyltoluene, 3,5-diethyltoluene, o-ethyltoluene, p-ethyltoluene, m-propyltoluene, 4-ethyl-m-xylene, dimethylnaphthalenes, ethylnaphthalene, 2,3-dimethylanthracene, 9-ethylanthracene, 2-methylanthracene, o-methylanthracene, 9,10-dimethylphenanthrene and 3-methyl-phenanthrene. Further examples of aromatic compounds include hexylbenzene, nonylbenzene, dodecylbenzene, pentadecylbenzene, hexyltoluene, nonyltoluene, dodecyltoluene and pentadecytoluene. In another embodiment, the aromatic compound includes hydrocarbons, such as benzene, naphthalene, anthracene, naphthacene, perylene, coronene and phenanthrene, for example.

The alkylating agent may include olefins (e.g., ethylene, propylene, butene and pentene), alcohols (e.g., methanol, ethanol, propanol, butanol and pentanol), aldehydes (e.g., formaldehyde, acetaldehyde, propionaldehyde, butyraldehyde and n-valeraldehyde) and/or alkyl halides (e.g., methyl chloride, ethyl chloride, propyl chloride, butyl chloride and pentyl chloride), for example. In one embodiment, the alkylating agent includes a mixture of light olefins, such as mixtures of ethylene, propylene, butene and/or pentenes, for example.

In addition to the aromatic compound and the alkylating agent, the input stream 102 may further include other compounds in minor amounts (e.g., sometimes referred to as poisons or inactive compounds,) such as C₇ aliphatic compounds and/or nonaromatic compounds, for example. In one embodiment, the input stream 102 includes less than about 3% of such compounds or less than about 1%, for example.

The alkylation system 104 generally includes a plurality of multi-stage reaction vessels, an embodiment of which is illustrated in FIG. 2. In one embodiment, the plurality of multi-stage reaction vessels include a plurality of operably connected catalyst beds, such beds containing an alkylation catalyst (not shown.) Such reaction vessels are generally liquid phase reactors operated at reactor temperatures and pressures sufficient to maintain the alkylation reaction in the liquid phase, i.e., the aromatic compound is in the liquid phase. Such temperatures and pressures are generally determined by individual process parameters. For example, the reaction vessel temperature may be from about 300° F. to about 650° F. or from about 400° F. to about 520° F., for example. The reaction vessel pressure may be about 3000 psig or less or about 1000 psig or less, for example.

In one embodiment, the space velocity of the reaction vessel within the alkylation system 104 is from about 10 liquid hourly space velocity (LHSV) to about 200 LHSV, based on the alkylating agent feed, or from about 50 LHSV to about 100 LHSV or from about 65 LHSV to about 85 LHSV.

The alkylation output 106 generally includes a second aromatic compound, for example.

The first separation system 108 may include any process or combination of processes known to one skilled in the art for the separation of aromatic compounds. For example, the first separation system 108 may include one or more distillation columns (not shown,) either in series or in parallel. The number of such columns may depend on the volume of the alkylation output 106 passing therethrough, for example.

The overhead fraction 110 from the first separation system 108 generally includes the first aromatic compound.

The second separation system 114 may include any process known to one skilled in the art, for example, one or more distillation columns (not shown), either in series or in parallel.

The overhead fraction 116 from the second separation system 114 generally includes the second aromatic compound, such as ethylbenzene, which may be recovered and used for any suitable purpose, such as the production of styrene, for example.

The bottoms fraction 118 from the second separation system 114 generally includes heavier aromatic compounds, such as polyethylbenzene, cumene and/or butylbenzene, for example.

The third separation system 115 generally includes any process known to one skilled in the art, for example, one or more distillation columns (not shown), either in series or in parallel.

In a specific embodiment, the overhead fraction 120 from the third separation system 115 may include diethylbenzene and liquid phase triethylbenzene, for example. The bottoms fraction 119 (e.g., heavies) may be recovered from the third separation system 115 for further processing and recovery (not shown).

The transalkylation system 121 generally includes one or more reaction vessels having a transalkylation catalyst disposed therein. The reaction vessels may include any reaction vessel, combination of reaction vessels and/or number of reaction vessels (either in parallel or in series) known to one skilled in the art.

The transalkylation output 124 generally includes the second aromatic compound, for example.

In one embodiment, the transalkylation system 121 is operated under liquid phase conditions. For example, the transalkylation system 121 may be operated at a temperature of from about 65° C. to about 290° C. and a pressure of about 600 psig or less. In another embodiment, the transalkylation system 121 is operated under vapor phase conditions, for example.

The transalkylation catalyst generally includes a molecular sieve catalyst, such as a zeolite Y catalyst, for example.

In a specific embodiment, the input stream 102 includes benzene and ethylene. The benzene may be supplied from a variety of sources, such as a fresh benzene source and/or a variety of recycle sources. As used herein, the term “fresh benzene source” refers to a source including at least about 95 wt. % benzene, at least about 98 wt. % benzene or at least about 99 wt. % benzene, for example. As used herein, the term “recycle” refers to an output of a system, such as an alkylation system and/or a dehydrogenation system, which is then returned as input to either that same system or another system the same process. In one embodiment, the molar ratio of benzene to ethylene in the input stream 102 may be from about 1:1 to about 30:1, or from about 1:1 to about 20:1 or from about 5:1 to about 15:1, for example.

In a specific embodiment, benzene is recovered through line 110 and recycled (not shown) as input to the alkylation system 104, while ethylbenzene and/or polyalkylated benzenes are recovered via line 112.

The alkylation system 104 generally includes an alkylation catalyst. The input stream, e.g., benzene/ethylene, contacts the alkylation catalyst during the alkylation reaction to form the alkylation output, e.g., ethylbenzene. In one embodiment, the alkylation catalyst is a molecular sieve catalyst that may be the same or different than the transalkylation catalyst. For example, the alkylation catalyst may be a zeolite beta or zeolite Y catalyst.

The zeolite beta may have a silica to alumina molar ratio (expressed as SiO₂/Al₂0₃) of from about 10 to about 200, or from about 20 to 50, for example. In one embodiment, the zeolite beta may have a low sodium content, e.g., less than about 0.2 wt. % expressed as Na₂O, or less than about 0.02 wt. %, for example. The sodium content may be reduced by any method known to one skilled in the art, such as through ion exchange, for example. The formation of zeolite beta is further described in U.S. Pat. No. 3,308,069 and U.S. Pat. No. 4,642,226, which are incorporated by reference herein. The formation of Zeolite Y is described in U.S. Pat. No. 4,185,040, which is incorporated by reference herein.

Unfortunately, alkylation catalyst systems generally experience deactivation requiring either regeneration or replacement. Additionally, alkylation processes generally require periodic maintenance. Both circumstances generally produce disruptions for liquid phase alkylation processes.

However, embodiments of the invention provide a process wherein continuous production during catalyst regeneration and maintenance may be achieved. For example, one reactor may be taken off-line for potential removal and regeneration of the catalyst, while the remaining reactor may remain on-line for production. The point of such removal will depend on specific system conditions, but is generally a predetermined set point (e.g., catalyst productivity or time.)

When removing the catalyst from the reactor for regeneration, it may be possible to replace the catalyst and place the reactor on-line while the removed/deactivated catalyst is regenerated. In such an embodiment, the cost of replacing the catalyst is large and therefore such catalyst should have a long life before regeneration. Embodiments of the invention unexpectedly provide a catalyst capable of production lives greater than that of conventional molecular sieve catalysts, especially when utilized in “swing reactor” systems.

In addition, an unexpected increase in catalyst regenerability may be gained by utilizing cerium beta catalysts in such systems. Conventional catalysts generally increase the catalyst costs when using swing reactors. However, it has been unexpectedly discovered that cerium beta catalyst may be regenerated to at least a substantial portion of their pre deactivation activity. Such unexpected regeneration provides for increased catalyst activity and/or longer run times between regeneration and/or replacement of the catalyst. In addition, it has been observed that the poison selectivity of the catalyst may be optimized by the amount of aluminum and cerium present in the cerium catalyst.

Therefore, specific embodiments of the invention generally utilize a cerium promoted zeolite catalyst as the alkylation catalyst. In one embodiment, the cerium promoted zeolite catalyst is a cerium promoted zeolite beta catalyst. The cerium promoted zeolite beta (e.g., cerium beta) catalyst may be formed from any zeolite catalyst known to one skilled in the art. For example, the cerium beta catalyst may include zeolite beta modified by the inclusion of cerium. Any method of modifying the zeolite beta catalyst with cerium may be used. For example, in one embodiment, the zeolite beta may be formed by mildly agitating a reaction mixture including an alkyl metal halide and an organic templating agent for a time sufficient to crystallize the reaction mixture and form the zeolite beta (e.g., from about 1 day to many months via hydrothermal digestion), for example. The alkyl metal halide may include silica, alumina, sodium or another alkyl metal oxide, for example. The hydrothermal digestion may occur at temperatures of from slightly below the boiling point of water at atmospheric pressure to about 170° C. at pressures equal to or greater than the vapor pressure of water at the temperature involved, for example.

When regeneration of any catalyst within the system is desired, the regeneration procedure generally includes processing the deactivated catalyst at high temperatures, although the regeneration may include any regeneration procedure known to one skilled in the art. As stated previously, the catalyst may be regenerated either in the reactor and may be removed from the reactor for regeneration. Such regeneration is known to one skilled in the art. However, a non-limiting illustrative embodiment of in-line regeneration is described below.

Once a reactor is taken off-line, the catalyst disposed therein may be purged. Off-stream reactor purging may be performed by contacting the catalyst in the off-line reactor with a purging stream, which may include any suitable inert gas (e.g., nitrogen), for example. The off-stream reactor purging conditions are generally determined by individual process parameters and are generally known to one skilled in the art.

The catalyst may then undergo regeneration. The regeneration conditions may be any conditions that are effective for at least partially reactivating the catalyst and are generally known to one skilled in the art. For example, regeneration may include heating the alkylation catalyst to a temperature or a series of temperatures, such as a regeneration temperature of from about 50° C. to about 200° C. above the purging or alkylation reaction temperature, for example.

In one specific non-limiting embodiment, the alkylation catalyst is heated to a first temperature (e.g., 700° F.) with a gas containing nitrogen and about 2% oxygen, for example, for a time sufficient to provide an output stream having an oxygen content of about 0.5%. The alkylation catalyst may then be heated to a second temperature for a time sufficient to provide an output stream having an oxygen content of about 2.0%. The second temperature may be about 50° F. greater than the first temperature, for example. The second temperature is generally about 950° F. or less, for example. The catalyst may further be held at the second temperature for a period of time, or at a third temperature that is greater than the second temperature, for example.

Upon catalyst regeneration, the reactor is ready to be placed on-line.

FIG. 2 illustrates an embodiment of an alkylation system 200. The alkylation system 200 generally includes a plurality of alkylation reactors, such as two alkylation reactors 202 and 204, operating in parallel (e.g., swing reactors.) One or both alkylation reactors 202 and 204, which may be the same type of reaction vessel, or, in certain embodiments, may be different types of reaction vessels, may be placed on line at the same time so that both reactors are in service at the same time. For example, only one alkylation reactor may be on line while the other is undergoing maintenance, such as catalyst regeneration. In one embodiment, the alkylation system 200 is configured so that the input stream is split equally to supply approximately the same input to each alkylation reactor 202 and 204. However, such flow rates will be determined by each individual system.

This mode of operation (e.g., swing reactors) may involve operation of the individual reactors at relatively lower space velocities for prolonged periods of time with periodic relatively short periods of operation at enhanced, relatively higher space velocities when one reactor is taken off-stream. By way of example, during normal operation of the system 200, with both reactors 202 and 204 on-line, the input 206 stream may be supplied to each reactor (e.g., via lines 208 and 210) to provide a reduced space velocity. When a reactor is taken off-line and the feed rate continues unabated, the space velocity for the remaining reactor may approximately double.

In a specific embodiment, one or more of the plurality of alkylation reactors may include a plurality of interconnected catalyst beds. The plurality of catalyst beds may include from 2 to 15 beds, or from 5 to 10 beds or, in specific embodiments, 5 or 8 beds, for example.

FIG. 3 illustrates a non-limiting embodiment of an alkylation reactor 302. The alkylation reactor 302 includes five series connected catalyst beds designated as beds A, B, C, D, and E. An input stream 304 (e.g., benzene/ethylene) is introduced to the reactor 302, passing through each of the catalyst beds to contact the alkylation catalyst and form the alkylation output 308. Additional alkylating agent may be supplied via lines 306a, 306b, 306c and 306d to the interstage locations in the reactor 302. Additional aromatic compound may also be introduced to the interstage locations via lines 310a, 310b and 310c, for example.

What is claimed is:

1. An alkylation system comprising:
an alkylation input stream comprising a first aromatic compound;
a plurality of reaction vessels having an alkylation catalyst disposed therein, each reaction vessel adapted to receive at least a portion of the alkylation input stream and contact the portion of the alkylation input stream with the alkylation catalyst to form a second aromatic compound, wherein the reaction vessels are adapted for liquid phase alkylation.

2. The alkylation system of claim 1, wherein the first aromatic compound comprises benzene and the second aromatic compound comprises ethylbenzene.

3. The alkylation system of claim 1, wherein the alkylation catalyst comprises a cerium promoted zeolite beta catalyst.

4. The alkylation system of claim 1, wherein the plurality of reaction vessels comprise two reaction vessels.

5. The alkylation system of claim 1 further comprising a separation system in operable communication with the one or more of the reaction vessels and adapted to receive the second aromatic compound.

6. An alkylation system comprising:
an alkylation system adapted to simultaneously alkylate a plurality of input streams under liquid phase conditions, wherein the input streams comprise an aromatic compound.

7. The alkylation system of claim 6 further comprising a plurality of catalyst beds having an alkylation catalyst disposed thereon.

8. The alkylation system of claim 7, wherein the alkylation catalyst is selected to minimize shutdown of the alkylation.

9. The alkylation system of claim 7, wherein the alkylation catalyst is capable of regeneration to a level that is within about 15 percent of its original activity level.

10. An alkylation method comprising:
providing an alkylation system comprising an alkylation catalyst disposed therein;
contacting a plurality of input streams with the alkylation catalyst to form an output stream, wherein the input streams comprise a first aromatic compound and an alkylating agent and the output stream comprises a second aromatic compound and wherein the first aromatic compound remains in liquid phase throughout the alkylation system.

11. The method of claim 10, wherein the first aromatic compound comprises benzene, the alkylating agent comprises ethylene and the second aromatic compound comprises ethylbenzene.

12. The method of claim 10, wherein the alkylation system comprises a plurality of reaction vessels adapted to receive the plurality of input streams.

13. The method of claim 12, wherein at least one of the plurality of input streams is passing through at least one of the reaction vessels while another of the reaction vessels is undergoing maintenance.

14. A regeneration method comprising:
exposing a first alkylation catalyst to an alkylation reaction environment within an alkylation system and terminating the exposure at a predetermined set point;
regenerating the first alkylation catalyst upon attaining the predetermined set point; and
simulatenously with the regenerating reacting an input stream with a second alkylation catalyst to form an output stream within the alkylation system, wherein the reaction is in the liquid phase.

15. The method of claim 14, wherein the first alkylation catalyst is regenerated in the alkylation system.

16. The method of claim 14 further comprising removing the first alkylation catalyst from the alkylation system prior to regeneration.

17. The method of claim 15, wherein a third alkylation catalyst is disposed within the alkylation system for production of an alkylation output during the regeneration of the first alkylation catalyst.

18. The method of claim 14, wherein the first alkylation catalyst is capable of regeneration within the alkylation system without system shutdown.
